(19) United States
(12) Patent Application Publication
(10) Pub. No.: US 2026/0241148 A1
(43) Pub. Date: Aug. 20, 2026

(54) MEDICAL ACCESSORY FOR USE WITH A SYRINGE

(71) Applicant: BOSTON SCIENTIFIC SCIMED, INC., Maple Grove, MN (US)

(72) Inventors: Adam David Grovender, Maple Grove, MN (US); Phil Litecky, Forest Lake, MN (US); Adam Troy Olson, Plymouth, MN (US); Eric T. Gagner, Minnetonka, MN (US)

(73) Assignee: BOSTON SCIENTIFIC SCIMED, INC., MAPLE GROVE, MN (US)

(21) Appl. No.: 19/539,890

(22) Filed: Feb. 13, 2026

Related U.S. Application Data
(60) Provisional application No. 63/759,346, filed on Feb. 17, 2025.

(51) Int. Cl. A61M 25/10

(52) U.S. Cl. CPC A61M 25/10182

(57) ABSTRACT

An accessory for use with a syringe includes a body portion configured to couple to a barrel of the syringe, and a proximal flange portion configured to engage with a finger flange of the syringe. An accessory includes a first portion including a first body portion configured to couple to the barrel and a first flange portion configured to engage with the finger flange, and a second portion including a second body portion configured to couple to the barrel and a second flange portion configured to engage with the finger flange. The first portion is hingedly attached to the second portion. A method includes coupling the accessory to a syringe, retracting a plunger of the syringe to fill the barrel with fluid, depressing the plunger to inflate a balloon, and subsequently retracting the plunger to deflate the balloon. The accessory prevents the plunger from exiting the barrel.

### CROSS-REFERENCE TO RELATED APPLICATIONS

This application claims the benefit of priority of U.S. Provisional Application No. 63/759,346 filed Feb. 17, 2025, the entire disclosure of which is hereby incorporated by reference.

### TECHNICAL FIELD

The present disclosure pertains to medical devices and uses thereof. More particularly, the present disclosure pertains to a medical accessory for use with a syringe in a procedure for inflation and deflation of a balloon, and associated methods.

### BACKGROUND

Inflatable balloons may be used in a variety of procedures including, but not limited to, balloon aortic valvuloplasty. Such procedures are typically performed with inexpensive and/or standard syringes of adequate volume to supply necessary inflation fluid to expand a balloon, but also possess enough dead space to provide sufficient deflation (via creating vacuum) when the plunger of the syringe is retracted. In some instances, volumetric size control of semi-compliant or non-complaint balloons requires a precise volume of fluid loaded into the syringe. Miscommunication and/or mis-reads of the syringe or reference chart(s) may result in an incorrect volume of fluid being loaded into the syringe. In some instances, the vacuum created during/for balloon deflation may be lost if the plunger is inadvertently removed from the syringe, which may make removal of the balloon more complicated.

Of the known medical devices and methods, each has certain advantages and disadvantages. There is an ongoing need to provide alternative medical devices as well as alternative methods for manufacturing and using medical devices.

### SUMMARY

In one example, a medical accessory for use with a syringe may comprise a body portion configured to releasably couple to a barrel of the syringe, and a proximal flange portion configured to engage with a finger flange of the syringe.

In addition, or alternatively, to any example disclosed herein, the body portion comprises a window configured to permit viewing of the barrel of the syringe through the body portion.

In addition, or alternatively, to any example disclosed herein, the body portion comprises a plurality of markings corresponding to different fluid volumes within the barrel of the syringe disposed along the window.

In addition, or alternatively, to any example disclosed herein, the different fluid volumes correspond to different balloon inflation sizes.

In addition, or alternatively, to any example disclosed herein, the body portion comprises a proximal portion extending distally from the proximal flange portion and a distal portion disposed distal of the window.

In addition, or alternatively, to any example disclosed herein, the body portion extends circumferentially around a central longitudinal axis.

In addition, or alternatively, to any example disclosed herein, the proximal portion extends a first angular distance around the central longitudinal axis and the distal portion extends a second angular distance around the central longitudinal axis greater than the first angular distance.

In addition, or alternatively, to any example disclosed herein, the first angular distance is greater than 180 degrees and less than 270 degrees.

In addition, or alternatively, to any example disclosed herein, the body portion is configured to snap onto the barrel of the syringe.

In addition, or alternatively, to any example disclosed herein, the body portion is configured to engage the syringe in a lateral direction.

In addition, or alternatively, to any example disclosed herein, the body portion comprises a C-shaped shell configured to engage a cylindrical outer surface of the barrel of the syringe.

In addition, or alternatively, to any example disclosed herein, the proximal flange portion is configured to engage the finger flange of syringe in a lateral direction.

In addition, or alternatively, to any example disclosed herein, the proximal flange portion is configured to slidably receive the finger flange of the syringe within an interior cavity of the proximal flange portion.

In addition, or alternatively, to any example disclosed herein, the proximal flange portion comprises a lateral extension extending from the proximal flange portion toward a central longitudinal axis of the body portion, the lateral extension being configured to prevent a plunger of the syringe from being removed from the barrel of the syringe.

In addition, or alternatively, to any example disclosed herein, and in a second example, a medical accessory for use with a syringe may comprise a first portion comprising a first body portion configured to releasably couple to a barrel of the syringe, and a first proximal flange portion configured to engage with a finger flange of the syringe, and a second portion comprising a second body portion configured to releasably couple to the barrel of the syringe, and a second proximal flange portion configured to engage with the finger flange of the syringe. The first portion maybe hingedly attached to the second portion.

In addition, or alternatively, to any example disclosed herein, the first proximal flange portion comprises a first lateral extension extending toward a central longitudinal axis of the medical accessory and the second proximal flange portion comprises a second lateral extension extending toward the central longitudinal axis of the medical accessory.

In addition, or alternatively, to any example disclosed herein, the first lateral extension and the second lateral extension are configured to prevent a plunger of the syringe from being removed from the barrel of the syringe.

In addition, or alternatively, to any example disclosed herein, the first body portion comprises a first window configured to permit viewing of the barrel of the syringe through the first body portion, and the second body portion comprises a second window configured to permit viewing of the barrel of the syringe through the second body portion.

In addition, or alternatively, to any example disclosed herein, the first window has a first length extending longitudinally along more than 50% of a length of the first body portion, and the second window has a second length extending longitudinally along more than 50% of a length of the second body portion.

In addition, or alternatively, to any example disclosed herein, the first body portion is configured to engage with the second body portion to collectively surround the barrel of the syringe.

In addition, or alternatively, to any example disclosed herein, and in a third example, a method of using a syringe in a procedure involving inflation and deflation of a balloon may comprise: releasably coupling a medical accessory to the syringe, the medical accessory comprising a body portion configured to releasably couple to a barrel of the syringe, and a proximal flange portion configured to engage with a finger flange of the syringe; with the medical accessory coupled to the barrel of the syringe, retracting a plunger disposed within the barrel of the syringe from a first position adjacent a distal end of the barrel of the syringe to a second position to fill the barrel of the syringe with an inflation fluid corresponding to an expanded size of the balloon; after filling the syringe with inflation fluid, and with the medical accessory still coupled to the barrel of the syringe, depressing the plunger of the syringe from the second position to the first position to inflate the balloon; and subsequently retracting the plunger of the syringe, with the medical accessory still coupled to the barrel of the syringe, to a proximal end of the barrel of the syringe to apply a vacuum to the balloon, thereby deflating the balloon. The medical accessory may be configured to prevent the plunger of the syringe from exiting the barrel of the syringe.

The above summary of some embodiments, aspects, and/or examples is not intended to describe each disclosed embodiment or every implementation of the present disclosure. The figures and detailed description which follow more particularly exemplify these embodiments .

### BRIEF DESCRIPTION OF THE DRAWINGS

The disclosure may be more completely understood in consideration of the following detailed description in connection with the accompanying drawings, in which:

FIG. 1 illustrates selected aspects of a syringe;

FIGS. 2-5 illustrate selected aspects of a medical accessory according to the disclosure;

FIGS. 6-7 illustrate selected aspects related to use of the medical accessory of FIGS. 2-5 with the syringe of FIG. 1;

FIG. 8 illustrates selected aspects of a medical accessory according to the disclosure;

FIG. 9 illustrates selected aspects related to use of the medical accessory of FIG. 8 with the syringe of FIG. 1;

FIG. 10 illustrates selected aspects of an alternative configuration of the medical accessory of FIGS. 8-9 according to the disclosure; and

FIGS. 11-13 illustrate selected aspects related to a method of using a syringe with the medical accessory of FIGS. 2-5 in a procedure involving inflation and deflation of a balloon.

While aspects of the disclosure are amenable to various modifications and alternative forms, specifics thereof have been shown by way of example in the drawings and will be described in detail. It should be understood, however, that the intention is not to limit aspects of the disclosure to the particular embodiments described. On the contrary, the intention is to cover all modifications, equivalents, and alternatives falling within the spirit and scope of the disclosure.

### DETAILED DESCRIPTION

The following description should be read with reference to the drawings, which are not necessarily to scale, wherein like reference numerals indicate like elements throughout the several views. The detailed description and drawings are intended to illustrate but not limit the disclosure. Those skilled in the art will recognize that the various elements described and/or shown may be arranged in various combinations and configurations without departing from the scope of the disclosure. The detailed description and drawings illustrate example embodiments of the disclosure.

For the following defined terms, these definitions shall be applied unless a different definition is given in the claims or elsewhere in this specification.

All numeric values are herein assumed to be modified by the term “about,” whether or not explicitly indicated. The term “about”, in the context of numeric values, generally refers to a range of numbers that one of skill in the art would consider equivalent to the recited value (e.g., having the same function or result). In many instances, the term “about” may include numbers that are rounded to the nearest significant figure. Other uses of the term “about” (e.g., in a context other than numeric values) may be assumed to have their ordinary and customary definition(s), as understood from and consistent with the context of the specification, unless otherwise specified.

The recitation of numerical ranges by endpoints includes all numbers within that range, including the endpoints (e.g., 1 to 5 includes 1, 1.5, 2, 2.75, 3, 3.80, 4, and 5).

Although some suitable dimensions, ranges, and/or values pertaining to various components, features and/or specifications are disclosed, one of skill in the art, incited by the present disclosure, would understand desired dimensions, ranges, and/or values may deviate from those expressly disclosed.

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise. It is to be noted that to facilitate understanding, certain features of the disclosure may be described in the singular, even though those features may be plural or recurring within the disclosed embodiment(s). Each instance of the features may include and/or be encompassed by the singular disclosure(s), unless expressly stated to the contrary. For example, a reference to one feature may be equally referred to all instances and quantities beyond one of said feature unless clearly stated to the contrary. As such, it will be understood that the following discussion may apply equally to any and/or all components for which there are more than one within the device, etc. unless explicitly stated to the contrary.

Relative terms such as “proximal”, “distal”, “advance”, “retract”, variants thereof, and the like, may be generally considered with respect to the positioning, direction, and/or operation of various elements relative to a user/operator/manipulator of the device, wherein “proximal” and “retract” indicate or refer to closer to or toward the user and “distal” and “advance” indicate or refer to farther from or away from the user. In some instances, the terms “proximal” and “distal” may be arbitrarily assigned to facilitate understanding of the disclosure, and such instances will be readily apparent to the skilled artisan. Other relative terms, such as “upstream”, “downstream”, “inflow”, and “outflow” refer to a direction of fluid flow within a lumen, such as a body lumen, a blood vessel, or within a device. Still other relative terms, such as “axial”, “circumferential”, “longitudinal”, “lateral”, “radial”, etc. and/or variants thereof generally refer to direction and/or orientation relative to a central longitudinal axis of the disclosed structure or device.

The term “extent” may be understood to mean the greatest measurement of a stated or identified dimension, unless the extent or dimension in question is preceded by or identified as a “minimum”, which may be understood to mean the smallest measurement of the stated or identified dimension. For example, “outer extent” may be understood to mean an outer dimension, “radial extent” may be understood to mean a radial dimension, “longitudinal extent” may be understood to mean a longitudinal dimension, etc. Each instance of an “extent” may be different (e.g., axial, longitudinal, lateral, radial, circumferential, etc.) and will be apparent to the skilled person from the context of the individual usage. Generally, an “extent” may be considered a greatest possible dimension measured according to the intended usage, while a “minimum extent” may be considered a smallest possible dimension measured according to the intended usage. In some instances, an “extent” may generally be measured orthogonally within a plane and/or cross-section, but may be, as will be apparent from the particular context, measured differently – such as, but not limited to, angularly, radially, circumferentially (e.g., along an arc), etc.

The terms “monolithic” and “unitary” shall generally refer to an element or elements made from or consisting of a single structure or base unit/element. A monolithic and/or unitary element shall exclude structure and/or features made by assembling or otherwise joining multiple discrete structures or elements together.

It is noted that references in the specification to “an embodiment”, “some embodiments”, “other embodiments”, etc., indicate that the embodiment(s) described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it would be within the knowledge of one skilled in the art to implement the particular feature, structure, or characteristic in connection with other embodiments, whether or not explicitly described, unless clearly stated to the contrary. That is, the various individual elements described below, even if not explicitly shown in a particular combination, are nevertheless contemplated as being combinable or arrangeable with each other to form other additional embodiments or to complement and/or enrich the described embodiment(s), as would be understood by one of ordinary skill in the art.

For the purpose of clarity, certain identifying numerical nomenclature (e.g., first, second, third, fourth, etc.) may be used throughout the description and/or claims to name and/or differentiate between various described and/or claimed features. It is to be understood that the numerical nomenclature is not intended to be limiting and is exemplary only. In some embodiments, alterations of and deviations from previously used numerical nomenclature may be made in the interest of brevity and clarity. That is, a feature identified as a “first” element may later be referred to as a “second” element, a “third” element, etc. or may be omitted entirely, and/or a different feature may be referred to as the “first” element. The meaning and/or designation in each instance will be apparent to the skilled practitioner.

Additionally, it should be noted that in any given figure, some features may not be shown, or may be shown schematically, for clarity and/or simplicity. Additional details regarding some components and/or method steps may be illustrated in other figures in greater detail. It is noted that some reference numbers may be discussed but are not expressly shown with respect to a particular figure. Reference numbers discussed but not expressly shown may be shown in other figures. Similarly, some reference numbers shown but not expressly discussed may be discussed with respect to other figures herein. The systems, devices, and/or methods disclosed herein may provide a number of desirable features and benefits as described in more detail below.

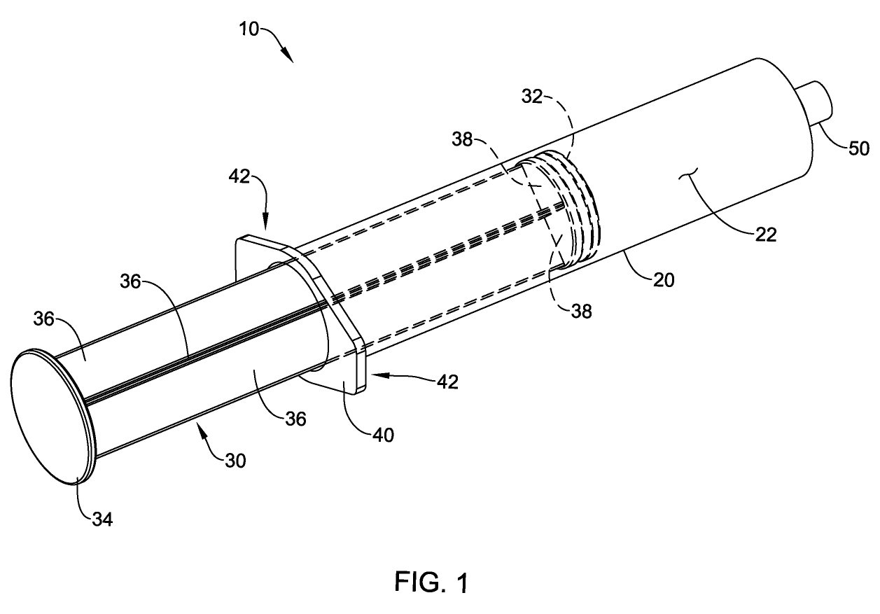

FIG. 1 illustrates selected aspects of a syringe 10 that may be used in a procedure involving inflation and deflation of a balloon. In one example, the syringe 10 may be used in a balloon aortic valvuloplasty (BAV) procedure. In another example, the syringe may be used to deploy a stent. Other configurations and/or procedures are also contemplated. The syringe 10 includes a barrel 20 and a plunger 30 slidably disposed within the barrel 20. The barrel 20 includes a cylindrical outer surface 22, and may include an inner surface that is cylindrical.

The plunger 30 includes a stopper 32 at its distal end. The stopper 32 is formed from rubber or another polymeric material to seal against the inner surface of the barrel 20. In instances where the inner surface is cylindrical, the stopper 32 has a substantially round and/or circular outer perimeter and/or shape. The barrel 20 is filled with fluid for inflating the balloon when the plunger 30 is depressed. The barrel 20 includes dead space therein (e.g., between the distal end of the plunger and a proximal end of the barrel 20) to create a vacuum for deflating the balloon when the plunger 30 is retracted.

The syringe 10 includes a finger flange 40 disposed at the proximal end of the barrel 20. The finger flange 40 typically extends in opposing directions from the barrel 20 so that the finger flange 40 may be engaged by at least two fingers of a hand of a user on opposite sides of the barrel 20. A user may place at least two fingers (of the same hand) on or against a distally facing surface 42 of the finger flange 40 and a thumb of the same hand against a thumb rest 34 of the plunger 30. The thumb rest 34 is disposed at a proximal end of the plunger 30. This allows the user to squeeze with his or her hand to depress the plunger 30 and eject fluid disposed within the barrel 20 through a distal hub 50. The distal hub 50 may be coupled to a needle, a Luer connector, a catheter, etc. as is known in the art.

The plunger 30 may include a plurality of longitudinal ribs 36 extending between the thumb rest 34 and a proximal face 38 of the stopper 32. The plurality of longitudinal ribs 36 may be joined together along and/or at a central longitudinal axis of the plunger 30. The plurality of longitudinal ribs 36 provides column strength to the plunger 30 for depressing the plunger 30 and/or ejecting the fluid from the barrel 20. For example, the plunger 30 may be used to generate fluid pressure within the balloon above one (1) atmosphere, and in some embodiments, the plunger 30 may be used to generate three (3) or more atmospheres of pressure within the balloon with a manual push of the plunger 30 (e.g., without mechanical assistance) by the hand of the user.

The syringe 10 is typically made from a polymeric material, such as polyethylene. Other materials are also contemplated. In some embodiments, the syringe 10 and/or at least the barrel 20 of the syringe 10 is formed from a material that is at least partially see-through (e.g., translucent, etc.) such that the stopper 32 may be seen within and/or through the barrel 20 of the syringe 10. In some embodiments, the syringe 10 and/or at least the barrel 20 of the syringe 10 is formed from a material that is completely see-through (e.g., transparent, etc.) such that the stopper 32 may be seen within and/or through the barrel 20 of the syringe 10.

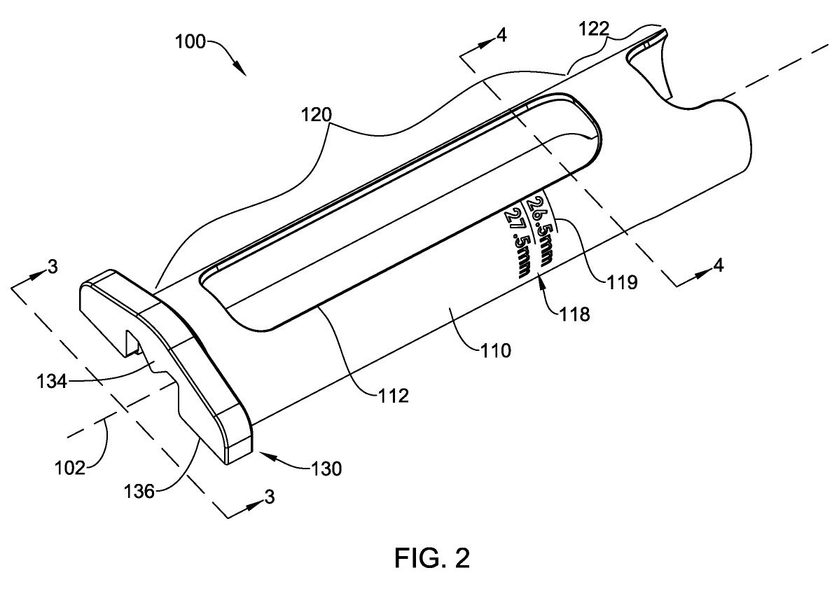

FIGS. 2-5 illustrate selected aspects of a medical accessory 100 for use with the syringe 10. It shall be understood that the medical accessory 100 is contemplated for use with any standard syringe, and is not expressly limited to the syringe 10 and/or the features thereof discussed herein. In some embodiments, the syringe 10 may be a 50 cubic centimeter (cc) syringe. In some embodiments, the syringe 10 may be a 30 cc syringe. Other configurations are also contemplated, and the medical accessory 100 may be manufactured in a variety of different sizes for use with varying sizes of syringes.

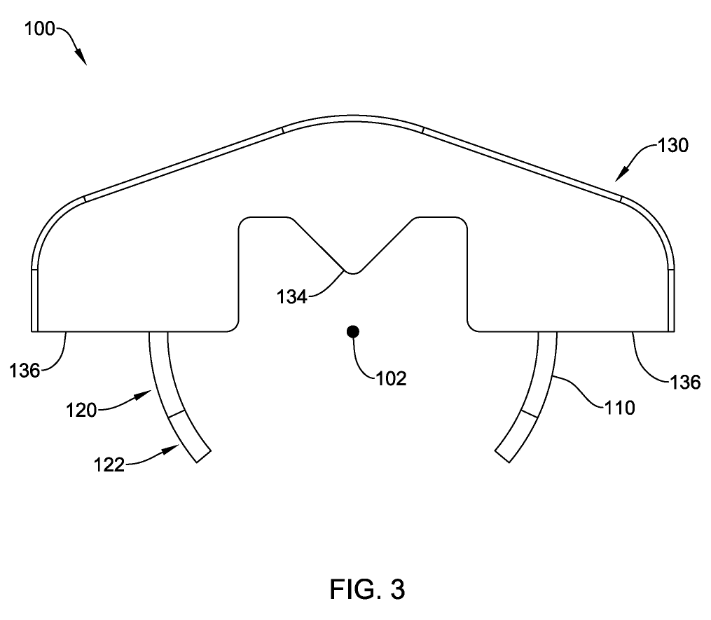

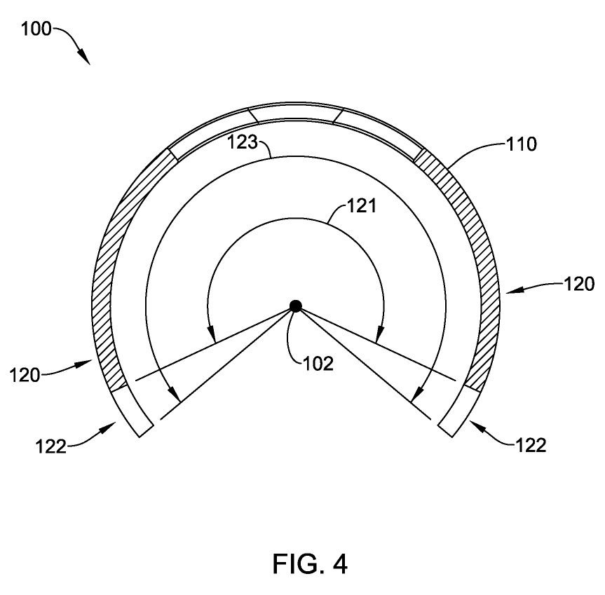

In some embodiments, the medical accessory 100 may comprise a body portion 110 configured to releasably engage with and/or releasably couple to the barrel 20 of the syringe 10. In some embodiments, the body portion 110 may comprise a window 112 configured to permit viewing of the barrel 20 of the syringe 10 through the body portion 110. In some embodiments, the body portion 110 may extend circumferentially around a central longitudinal axis 102 of the medical accessory 100 and/or the body portion 110, as seen more clearly in the end view of FIG. 3, taken along the line 3-3 in FIG. 2, and/or the cross-sectional view of FIG. 4, taken along the line 4-4 in FIG. 2. In some embodiments, the body portion 110 may comprise a C-shaped shell configured to engage the barrel 20 of the syringe 10.

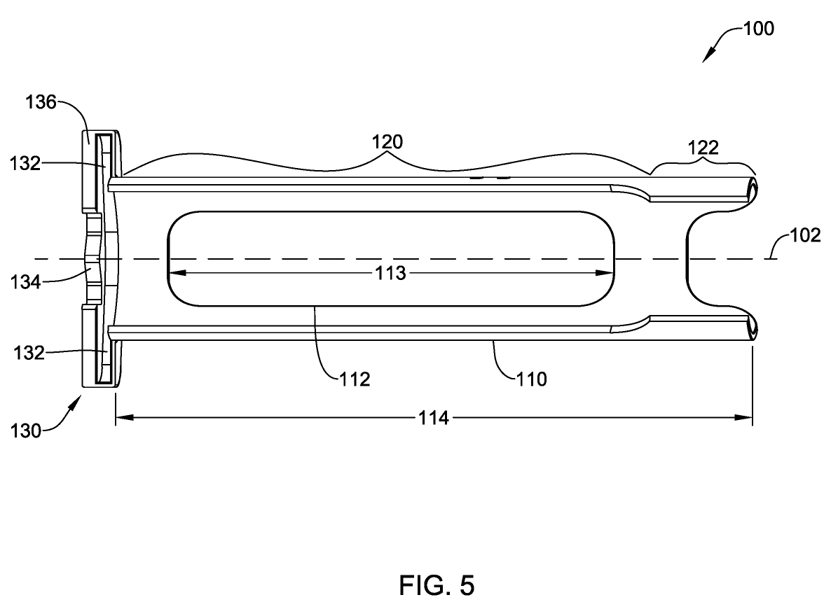

In some embodiments, the medical accessory 100 may comprise a proximal flange portion 130 configured to engage with the finger flange 40 of the syringe 10. In some embodiments, the proximal flange portion 130 may comprise an interior cavity 132 (e.g., FIG. 5). In some embodiments, the proximal flange portion 130 may comprise a lateral extension 134 extending from the proximal flange portion 130 toward the central longitudinal axis 102 of the medical accessory 100 and/or the body portion 110, as seen in FIG. 3. In some embodiments, the proximal flange portion 130 and/or the interior cavity 132 may extend laterally in opposite directions from a plane containing the central longitudinal axis 102 of the medical accessory 100 and/or the body portion 110.

In some embodiments, the proximal flange portion 130 may comprise a lateral edge 136. In some embodiments, the lateral edge 136 may extend along a line perpendicular to the central longitudinal axis 102 of the medical accessory 100 and/or the body portion 110. In some embodiments, the lateral edge 136 may extend in and/or along a plane containing the central longitudinal axis 102 of the medical accessory 100 and/or the body portion 110. In some alternative embodiments, the lateral edge 136 may be offset from and/or parallel to the plane containing the central longitudinal axis 102 of the medical accessory 100 and/or the body portion 110. Other configurations are also contemplated.

In some embodiments, the body portion 110 may comprise a proximal portion 120 extending distally from the proximal flange portion 130 and a distal portion 122 disposed distal of the window 112, as seen in FIGS. 2 and 5. In some embodiments, the proximal portion 120 may extend from the proximal flange portion 130 to the distal portion 122. In at least some embodiments, the proximal portion 120 may include and/or may contain the window 112. In some embodiments, the proximal portion 120 may extend distal of the window 112 such that the window 112 is entirely contained within the proximal portion 120. Other configurations are also contemplated.

In some embodiments, the proximal portion 120 may extend a first angular distance 121 around the central longitudinal axis 102 of the medical accessory 100 and/or the body portion 110, and the distal portion 122 may extend a second angular distance 123 around the central longitudinal axis 102 of the medical accessory 100 and/or the body portion 110, as seen in FIG. 4. In at least some embodiments, the second angular distance 123 may be greater than the first angular distance 121. In some embodiments, the first angular distance 121 may be greater than 180 degrees and less than 270 degrees. Other configurations are also contemplated.

In some embodiments, the window 112 may have a length 113 (e.g., FIG. 5) extending longitudinally along more than 50% of a length 114 (e.g., FIG. 5) of the body portion 110. In some embodiments, the window 112 may have a length 113 extending longitudinally along more than 60% of a length 114 of the body portion 110. In some embodiments, the window 112 may have a length 113 extending longitudinally along more than 70% of a length 114 of the body portion 110. In some embodiments, the window 112 may have a length 113 extending longitudinally along more than 75% of a length 114 of the body portion 110. In some embodiments, the window 112 may have a length 113 extending longitudinally along more than 80% of a length 114 of the body portion 110. Other configurations are also contemplated.

In some embodiments, the window 112 may be configured to permit viewing of a majority of the barrel 20 of the syringe 10, and/or the stopper 32 disposed therein, through the body portion 110. In some embodiments, the window 112 may be configured to permit viewing of at least 50% of the barrel 20 of the syringe 10 through the body portion 110. In some embodiments, the window 112 may be configured to permit viewing of at least 60% of the barrel 20 of the syringe 10 through the body portion 110. In some embodiments, the window 112 may be configured to permit viewing of at least 70% of the barrel 20 of the syringe 10 through the body portion 110. In some embodiments, the window 112 may be configured to permit viewing of at least 75% of the barrel 20 of the syringe 10 through the body portion 110. Other configurations are also contemplated.

In some embodiments, the body portion 110 may comprise a plurality of markings 118 corresponding to different fluid volumes within the barrel 20 of the syringe 10 disposed along the window 112. In some embodiments, the plurality of markings 118 may comprise units of volume, such as cubic centimeters (cc) or milliliters (ml). In at least some embodiments, the different fluid volumes may correspond to different balloon inflation sizes and/or diameters. For example, the plurality of markings 118 may comprise expanded sizes and/or diameters of a balloon (e.g., 23 millimeters (mm), 24 mm, 25 mm, 26 mm, 26.5 mm, 27.0 mm, 27.5 mm, 28 mm, 29 mm, 30 mm, etc.) Other configurations are also contemplated. In some embodiments, the different fluid volumes within the barrel 20 of the syringe 10 are fluid volumes necessary to expand and/or inflate a balloon to an expanded size and/or an inflated configuration having a specific size. In some embodiments, the plurality of markings 118 may comprise lines 119 printed on or formed in the body portion 110. In some alternative embodiments, the plurality of markings 118 may comprise arrows pointing at the window 112. In some further alternative embodiments, the plurality of markings 118 may comprise pointers or extensions extending into the window 112. Other configurations, including combinations thereof, are also contemplated.

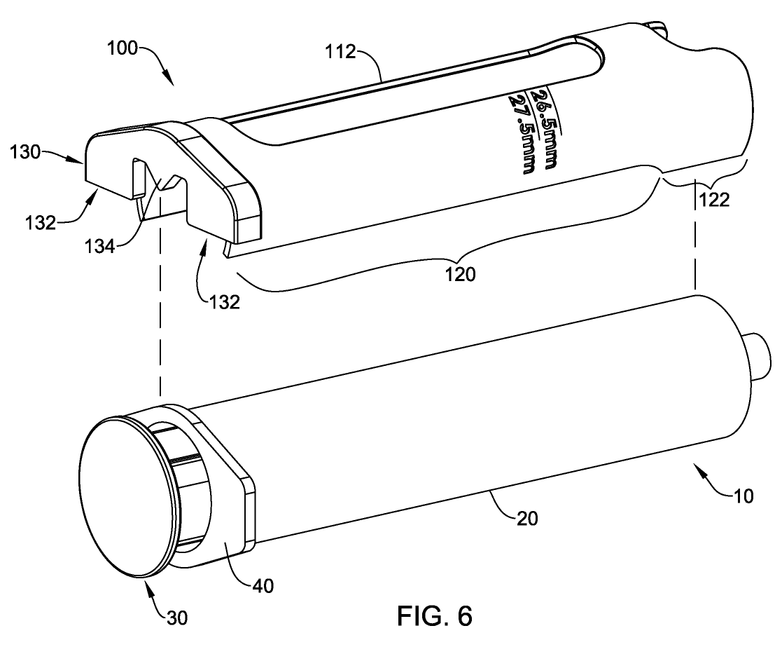

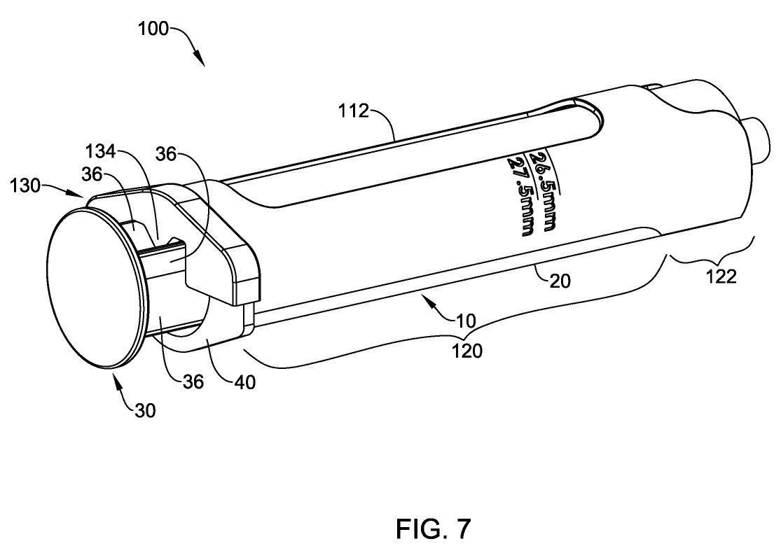

As noted above, the body portion 110 of the medical accessory 100 may be configured to releasably engage with and/or releasably couple to the barrel 20 of the syringe 10. In some embodiments, the body portion 110 of the medical accessory 100 may be configured to snap onto the barrel 20 of the syringe 10. Other configurations are also contemplated. In some embodiments, the body portion 110 of the medical accessory 100 may be configured to releasably engage with and/or releasably couple to the barrel 20 of the syringe 10 in a lateral direction, as seen in FIGS. 6-7. In some embodiments, the body portion 110 of the medical accessory 100 may comprise a C-shaped shell (see, for example, FIGS. 3-4) configured to releasably engage with and/or releasably couple to the cylindrical outer surface 22 of the barrel 20 of the syringe 10. In at least some embodiments, the C-shaped shell may be configured to matingly engage with the cylindrical outer surface 22 of the barrel 20 of the syringe 10 such that an inner surface of the body portion 110 of the medical accessory 100 is disposed against and/or in direct contact with the cylindrical outer surface 22 of the barrel of the syringe 10.

In some embodiments, the proximal flange portion 130 of the medical accessory 100 may be configured to releasably engage the finger flange 40 of the syringe 10 in a lateral direction (e.g., in the lateral direction that the body portion 110 of the medical accessory 100 is configured to releasably engage with and/or releasably couple to the barrel 20 of the syringe 10). In some embodiments, the proximal flange portion 130 of the medical accessory 100 may be configured to slidably receive the finger flange 40 of the syringe 10 within the interior cavity 132 of the proximal flange portion 130 of the medical accessory 100. Engagement of the finger flange 40 of the syringe 10 with the interior cavity 132 of the proximal flange portion 130 of the medical accessory 100 may prevent relative axial movement between the medical accessory 100 and the syringe 10. In some embodiments, the finger flange 40 of the syringe 10 may be captured and/or trapped within the interior cavity 132 of the proximal flange portion 130, thereby preventing relative axial movement therebetween.

As discussed herein, the proximal flange portion 130 may comprise the lateral extension 134 extending from the proximal flange portion 130 toward the central longitudinal axis 102 of the medical accessory 100 and/or the body portion 110, as shown in FIGS. 2-3. In some embodiments, the lateral extension 134 may extend between two adjacent longitudinal ribs of the plurality of longitudinal ribs 36 of the plunger 30, as seen in FIG. 7. In some embodiments, the lateral extension 134 may be configured to prevent the plunger 30 of the syringe 10 from being removed from the barrel 20 of the syringe 10 when the body portion 110 of the medical accessory 100 is engaged with and/or coupled to the barrel 20 of the syringe 10. In some embodiments, the lateral extension 134 may form a hard stop for the plunger 30 when the proximal face 38 of the stopper 32 engages a distal face of the lateral extension 134, thereby preventing the plunger 30 from being completely withdrawn and/or removed from the barrel 20 of the syringe 10. Removal of the plunger 30 from the barrel 20 of the syringe 10 during a procedure may cause a loss of vacuum when deflating a balloon and/or when removing a balloon from a treatment site. As such, removal of the plunger 30 from the barrel 20 during the procedure is usually undesirable.

In some embodiments, the distal portion 122 of the body portion 110 of the medical accessory 100 may extend around more of a circumference of the barrel 20 of the syringe 10 than the proximal portion 120 of the body portion 110 of the medical accessory 100. In some embodiments, the distal portion 122 of the body portion 110 of the medical accessory 100 may form a securement element configured to secure the body portion 110 of the medical accessory 100 to the barrel 20 of the syringe 10. In some embodiments, the securement element may be configured to prevent the body portion 110 of the medical accessory 100 from freely falling off the barrel 20 of the syringe 10.

As discussed above, the window 112 of the body portion 110 of the medical accessory 100 may be configured to permit viewing of the barrel 20 of the syringe 10 through the body portion 110 of the medical accessory 100. In at least some embodiments, the window 112 of the body portion 110 of the medical accessory 100 may be configured to permit viewing of the stopper 32 within and/or through the barrel 20 of the syringe 10 through the body portion 110 of the medical accessory 100.

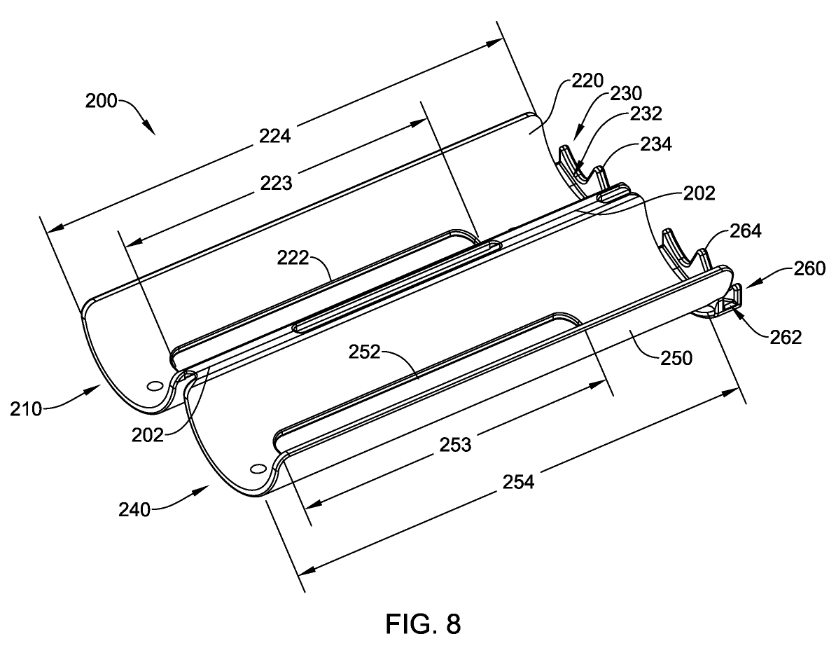

FIGS. 8-10 illustrate selected aspects of a medical accessory 200 for use with the syringe 10. It shall be understood that the medical accessory 200 is contemplated for use with any standard syringe, and is not expressly limited to the syringe 10 and/or the features thereof discussed herein. In some embodiments, the syringe 10 may be a 50 cubic centimeter (cc) syringe. In some embodiments, the syringe 10 may be a 30 cc syringe. Other configurations are also contemplated, and the medical accessory 200 may be manufactured in a variety of different sizes for use with varying sizes of syringes.

In some embodiments, the medical accessory 200 may comprise a first portion 210 and a second portion 240. In at least some embodiments, the first portion 210 may be hingedly attached to the second portion 240. In some embodiments, the first portion 210 may be hingedly attached to the second portion 240 with a living hinge 202, such that the first portion 210 is monolithically formed with the second portion 240 and the living hinge 202. In some embodiments, the first portion 210 may be hingedly attached to the second portion 240 with a pin-type hinge (e.g., a piano hinge, a butt or mortise hinge, etc.). Other configurations are also contemplated.

In some embodiments, the first portion 210 may comprise a first body portion 220 configured to releasably engage with and/or releasably couple to the barrel 20 of the syringe 10. In some embodiments, the first body portion 220 may comprise a first window 222 configured to permit viewing of the barrel 20 of the syringe 10 through the first body portion 220 when the medical accessory 200 is disposed in a closed position. In some embodiments, the first body portion 220 may extend circumferentially around a central longitudinal axis of the medical accessory 200 when the medical accessory 200 is disposed in the closed position.

In some embodiments, the first window 222 may have a first length 223 extending longitudinally along more than 50% of a length 224 of the first body portion 220. In some embodiments, the first window 222 may have a first length 223 extending longitudinally along more than 60% of a length 224 of the first body portion 220. In some embodiments, the first window 222 may have a first length 223 extending longitudinally along more than 70% of a length 224 of the first body portion 220. In some embodiments, the first window 222 may have a first length 223 extending longitudinally along more than 75% of a length 224 of the first body portion 220. In some embodiments, the first window 222 may have a first length 223 extending longitudinally along more than 80% of a length 224 of the first body portion 220. Other configurations are also contemplated.

In some embodiments, the first window 222 may be configured to permit viewing of a majority of the barrel 20 of the syringe 10, and/or the stopper 32 disposed therein, through the first body portion 220 when the medical accessory 200 is disposed in the closed position. In some embodiments, the first window 222 may be configured to permit viewing of at least 50% of the barrel 20 of the syringe 10 through the first body portion 220 when the medical accessory 200 is disposed in the closed position. In some embodiments, the first window 222 may be configured to permit viewing of at least 60% of the barrel 20 of the syringe 10 through the first body portion 220 when the medical accessory 200 is disposed in the closed position. In some embodiments, the first window 222 may be configured to permit viewing of at least 70% of the barrel 20 of the syringe 10 through the first body portion 220 when the medical accessory 200 is disposed in the closed position. In some embodiments, the first window 222 may be configured to permit viewing of at least 75% of the barrel 20 of the syringe 10 through the first body portion 220 when the medical accessory 200 is disposed in the closed position. Other configurations are also contemplated.

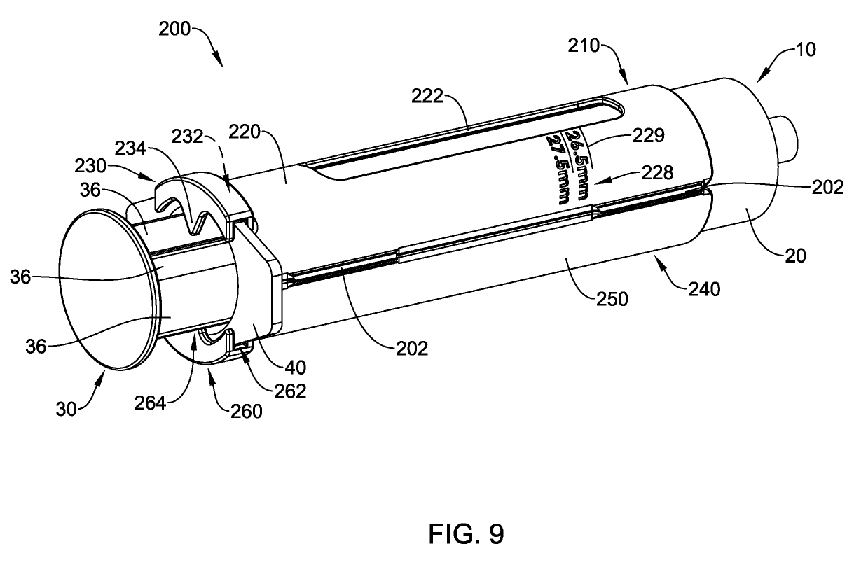

In some embodiments, the first body portion 220 may comprise a plurality of markings 228 (e.g., FIG. 9) corresponding to different fluid volumes within the barrel 20 of the syringe 10 disposed along the first window 222. In some embodiments, the plurality of markings 228 may comprise units of volume, such as cubic centimeters (cc) or milliliters (ml). In at least some embodiments, the different fluid volumes may correspond to different balloon inflation sizes and/or diameters. For example, the plurality of markings 228 (e.g., FIG. 9) may comprise expanded sizes and/or diameters of a balloon (e.g., 23 millimeters (mm), 24 mm, 25 mm, 26 mm, 26.5 mm, 27.0 mm, 27.5 mm, 28 mm, 29 mm, 30 mm, etc.) Other configurations are also contemplated. In some embodiments, the plurality of markings 228 may comprise lines 229 printed on or formed in the first body portion 220. In some alternative embodiments, the plurality of markings 228 may comprise arrows pointing at the first window 222. In some further alternative embodiments, the plurality of markings 228 may comprise pointers or extensions extending into the first window 222. Other configurations, including combinations thereof, are also contemplated.

In some embodiments, the first portion 210 may comprise a first proximal flange portion 230 configured to engage with the finger flange 40 of the syringe 10. In some embodiments, the first proximal flange portion 230 may comprise a first interior cavity 232. In some embodiments, the first proximal flange portion 230 may comprise a first lateral extension 234 extending from the first proximal flange portion 230 toward the central longitudinal axis of the medical accessory 200 when the medical accessory 200 is disposed in the closed position. In some embodiments, the first proximal flange portion 230 and/or the first interior cavity 232 may extend laterally in opposite directions from a plane containing the central longitudinal axis of the medical accessory 200.

In some embodiments, the second portion 240 may comprise a second body portion 250 configured to releasably engage with and/or releasably couple to the barrel 20 of the syringe 10. In some embodiments, the second body portion 250 may comprise a second window 252 configured to permit viewing of the barrel 20 of the syringe 10 through the first body portion 220 when the medical accessory 200 is disposed in the closed position. In some embodiments, the second body portion 250 may extend circumferentially around the central longitudinal axis of the medical accessory 200 when the medical accessory 200 is disposed in the closed position.

In some embodiments, the second window 252 may have a second length 253 extending longitudinally along more than 50% of a length 254 of the second body portion 250. In some embodiments, the second window 252 may have a second length 253 extending longitudinally along more than 60% of a length 254 of the second body portion 250. In some embodiments, the second window 252 may have a second length 253 extending longitudinally along more than 70% of a length 254 of the second body portion 250. In some embodiments, the second window 252 may have a second length 253 extending longitudinally along more than 75% of a length 254 of the second body portion 250. In some embodiments, the second window 252 may have a second length 253 extending longitudinally along more than 80% of a length 254 of the second body portion 250. Other configurations are also contemplated.

In some embodiments, the second window 252 may be configured to permit viewing of a majority of the barrel 20 of the syringe 10, and/or the stopper 32 disposed therein, through the second body portion 250 when the medical accessory 200 is disposed in the closed position. In some embodiments, the second window 252 may be configured to permit viewing of at least 50% of the barrel 20 of the syringe 10 through the second body portion 250 when the medical accessory 200 is disposed in the closed position. In some embodiments, the second window 252 may be configured to permit viewing of at least 60% of the barrel 20 of the syringe 10 through the second body portion 250 when the medical accessory 200 is disposed in the closed position. In some embodiments, the second window 252 may be configured to permit viewing of at least 70% of the barrel 20 of the syringe 10 through the second body portion 250 when the medical accessory 200 is disposed in the closed position. In some embodiments, the second window 252 may be configured to permit viewing of at least 75% of the barrel 20 of the syringe 10 through the second body portion 250 when the medical accessory 200 is disposed in the closed position. Other configurations are also contemplated.

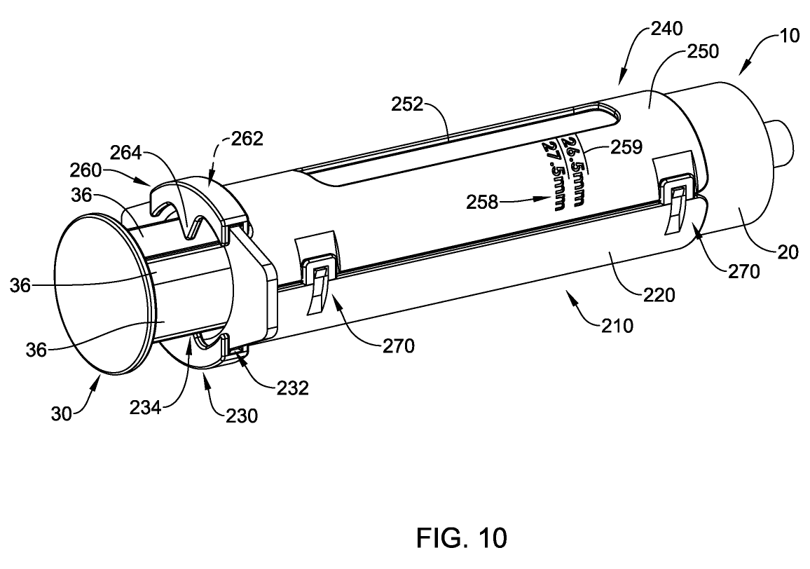

In some embodiments, the second body portion 250 may optionally comprise a second plurality of markings 258 (e.g., FIG. 10) corresponding to different fluid volumes within the barrel 20 of the syringe 10 disposed along the second window 252. In some embodiments, the second plurality of markings 258 may comprise units of volume, such as cubic centimeters (cc) or milliliters (ml). In at least some embodiments, the different fluid volumes may correspond to different balloon inflation sizes and/or diameters. For example, the second plurality of markings 258 may comprise expanded sizes and/or diameters of a balloon (e.g., 23 millimeters (mm), 24 mm, 25 mm, 26 mm, 26.5 mm, 27.0 mm, 27.5 mm, 28 mm, 29 mm, 30 mm, etc.) Other configurations are also contemplated. In some embodiments, the different fluid volumes within the barrel 20 of the syringe 10 are fluid volumes necessary to expand and/or inflate a balloon to an expanded size and/or an inflated configuration having a specific size. In some embodiments, the second plurality of markings 258 may comprise lines 259 (e.g., FIG. 10) printed on or formed in the second body portion 250. In some alternative embodiments, the second plurality of markings 258 may comprise arrows pointing at the second window 252. In some further alternative embodiments, the second plurality of markings 258 may comprise pointers or extensions extending into the second window 252. Other configurations, including combinations thereof, are also contemplated.

In some embodiments, the second portion 240 may comprise a second proximal flange portion 260 configured to engage with the finger flange 40 of the syringe 10. In some embodiments, the second proximal flange portion 260 may comprise a second interior cavity 262. In some embodiments, the second proximal flange portion 260 may comprise a second lateral extension 264 extending from the second proximal flange portion 260 toward the central longitudinal axis of the medical accessory 200 when the medical accessory 200 is disposed in the closed position. In some embodiments, the second proximal flange portion 260 and/or the second interior cavity 262 may extend laterally in opposite directions from a plane containing the central longitudinal axis of the medical accessory 200.

As noted above, the first body portion 220 and/or the second body portion 250 of the medical accessory 200 may be configured to releasably engage with and/or releasably couple to the barrel 20 of the syringe 10. In some embodiments, the first body portion 220 and/or the second body portion 250 of the medical accessory 200 may be configured to close around the barrel 20 of the syringe 10. Other configurations are also contemplated. In some embodiments, the first body portion 220 and/or the second body portion 250 of the medical accessory 200 may be configured to releasably engage with and/or releasably couple to the barrel 20 of the syringe 10 in a lateral direction. In some embodiments, the first body portion 220 and/or the second body portion 250 of the medical accessory 200 may each comprise a C-shaped shell configured to releasably engage with and/or releasably couple to the cylindrical outer surface 22 of the barrel 20 of the syringe 10. In at least some embodiments, the first body portion 220 and/or the second body portion 250 may be configured to matingly engage with the cylindrical outer surface 22 of the barrel 20 of the syringe 10 such that an inner surface of the first body portion 220 and/or an inner surface of the second body portion 250 of the medical accessory 200 is disposed against and/or in direct contact with the cylindrical outer surface 22 of the barrel of the syringe 10 when the medical accessory 200 is disposed in the closed position.

In some embodiments, the first body portion 220 may be configured to engage with the second body portion 250 to collectively surround the barrel 20 of the syringe 10 when the medical accessory 200 is disposed in the closed position, as seen in FIG. 9. In some embodiments, a first free edge of the first body portion 220, wherein the first free edge is disposed opposite the living hinge 202, may be configured to move towards, engage with, and/or contact a second free edge of the second body portion 250, wherein the second free edge is disposed opposite the living hinge 202, when the first body portion 220 and the second body portion 250 are releasably coupled to the barrel 20 of the syringe 10 to thereby surround the barrel 20 of the syringe 10 in the closed position. In some embodiments, the first body portion 220 and/or the second body portion 250 may be biased toward the closed position. In some embodiments, the first body portion 220 and/or the second body portion 250 may be biased toward the closed position after being partially shifted towards the closed position. In some embodiments, the first body portion 220 and/or the second body portion 250 may be self-biased toward the closed position. Other configurations are also contemplated. In some embodiments, the first body portion 220 and/or the second body portion 250 may be self-biased toward the closed position after being partially shifted towards the closed position. Other configurations are also contemplated.

In some embodiments, the first proximal flange portion 230 and/or the second proximal flange portion 260 of the medical accessory 200 may be configured to releasably engage the finger flange 40 of the syringe 10 in a lateral direction (e.g., in the lateral direction(s) that the first body portion 220 and/or the second body portion 250 of the medical accessory 200 is configured to releasably engage with and/or releasably couple to the barrel 20 of the syringe 10, respectively). In some embodiments, the first proximal flange portion 230 of the medical accessory 200 may be configured to slidably receive the finger flange 40 of the syringe 10 within the first interior cavity 232 of the first proximal flange portion 230 of the medical accessory 100. In some embodiments, the second proximal flange portion 260 of the medical accessory 200 may be configured to slidably receive the finger flange 40 of the syringe 10 within the second interior cavity 262 of the second proximal flange portion 260 of the medical accessory 100. Engagement of the finger flange 40 of the syringe 10 with the first interior cavity 232 of the first proximal flange portion 230 and/or the second interior cavity 262 of the second proximal flange portion 260 of the medical accessory 200 may prevent relative axial movement between the medical accessory 200 and the syringe 10. In some embodiments, the finger flange 40 of the syringe 10 may be captured and/or trapped within the first interior cavity 232 of the first proximal flange portion 230 and the second interior cavity 262 of the second proximal flange portion 260 when the medical accessory 200 is disposed in the closed position, thereby preventing relative axial movement therebetween.

In some embodiments, the first body portion 220 and/or the second body portion 250 may independently or collectively comprise at least one latching mechanism 270, as seen in FIG. 10. In some embodiments, the first body portion 220 may comprise a first portion of the at least one latching mechanism 270 and the second body portion 250 may comprise a second portion of the at least one latching mechanism 270. In some embodiments, the at least one latching mechanism 270 may be configured to secure the first body portion 220 to the second body portion 250 opposite the living hinge 202 (or pin-type hinge, where present) in the closed position. The first portion of the at least one latching mechanism 270 and the second portion of the at least one latching mechanism 270 may cooperate to secure the first body portion 220 to the second body portion 250 in the closed position. In some embodiments, the at least one latching mechanism 270 may comprise one or more snap elements. Other configurations are also contemplated.

As discussed herein, the first proximal flange portion 230 may comprise the first lateral extension 234 extending from the first proximal flange portion 230 toward the central longitudinal axis of the medical accessory 200 in the closed position, and the second proximal flange portion 260 may comprise the second lateral extension 264 extending from the second proximal flange portion 260 toward the central longitudinal axis of the medical accessory 200 in the closed position, as shown in FIGS. 9-10. In some embodiments, the first lateral extension 234 and/or the second lateral extension 264 may extend between two adjacent longitudinal ribs of the plurality of longitudinal ribs 36 of the plunger 30 when the medical accessory 200 is in the closed position. In some embodiments, the first lateral extension 234 and/or the second lateral extension 264 may be configured to prevent the plunger 30 of the syringe 10 from being removed from the barrel 20 of the syringe 10 when the first body portion 220 and the second body portion 250 of the medical accessory 200 are releasably engaged with and/or releasably coupled to the barrel 20 of the syringe 10 in the closed position. In some embodiments, the first lateral extension 234 and/or the second lateral extension 264 may form a hard stop for the plunger 30 when the proximal face 38 of the stopper 32 engages a distal face of the first lateral extension 234 and/or the second lateral extension 264, thereby preventing the plunger 30 from being completely withdrawn and/or removed from the barrel 20 of the syringe 10. Removal of the plunger 30 from the barrel 20 of the syringe 10 during a procedure may cause a loss of vacuum when deflating a balloon and/or when removing a balloon from a treatment site. As such, removal of the plunger 30 from the barrel 20 during the procedure is usually undesirable.

As discussed above, the first window 222 of the first body portion 220 and/or the second window 252 of the second body portion 250 of the medical accessory 200 may be configured to permit viewing of the barrel 20 of the syringe 10 through the first body portion 220 and/or the second body portion 250 of the medical accessory 200 in the closed position. In at least some embodiments, the first window 222 of the first body portion 220 and/or the second window 252 of the second body portion 250 of the medical accessory 200 may be configured to permit viewing of the stopper 32 within and/or through the barrel 20 of the syringe 10 through the first body portion 220 and/or the second body portion 250 of the medical accessory 200 in the closed position.

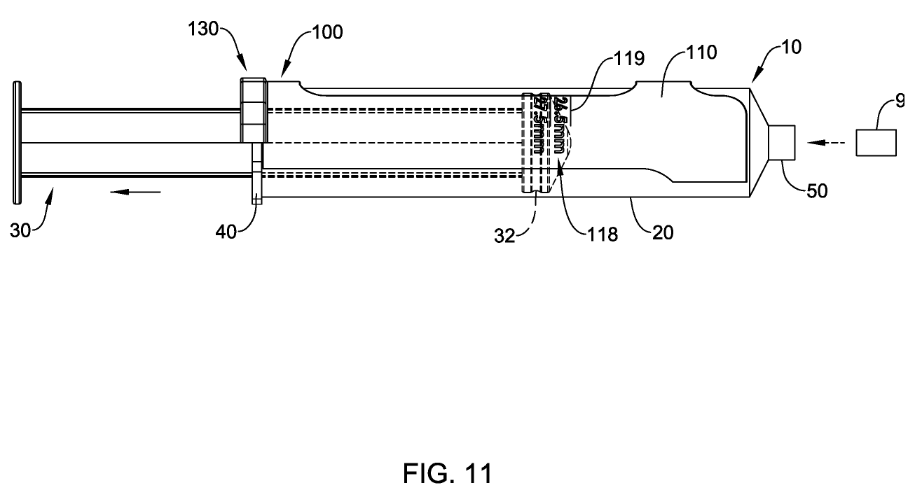

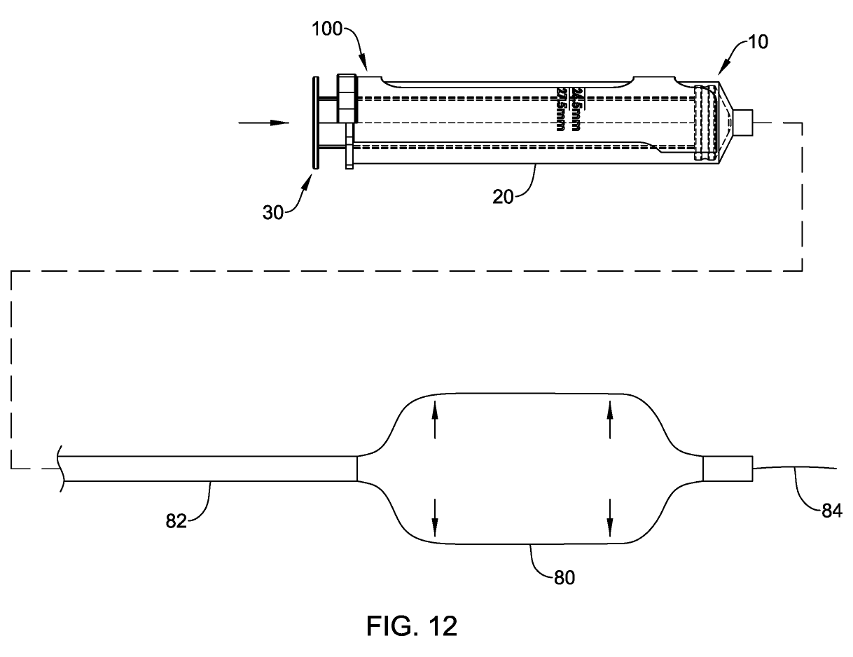

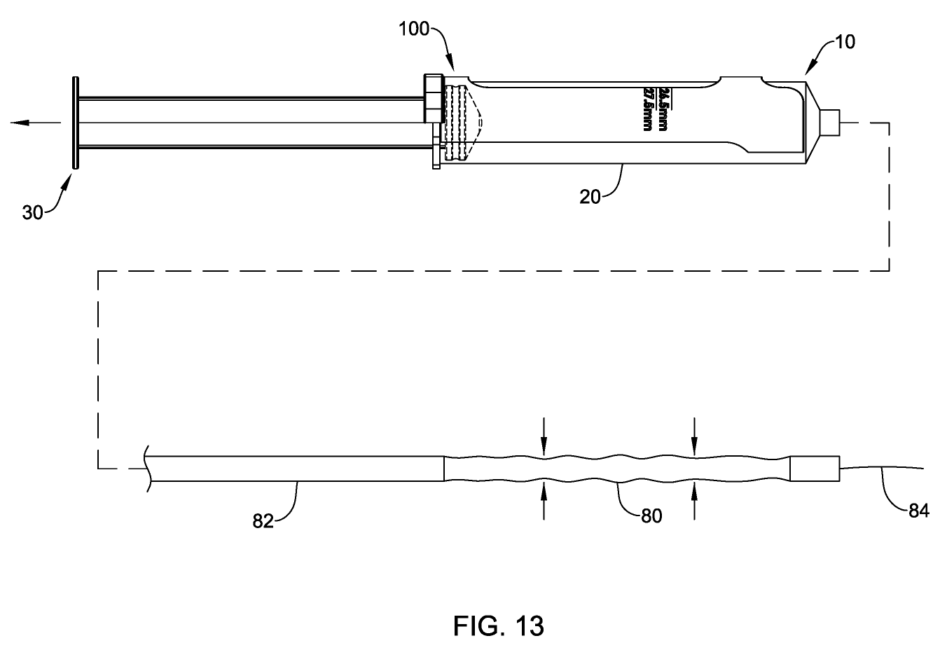

FIGS. 11-13 illustrate selected aspects of a method of using the syringe 10 in a procedure involving inflation and deflation of a balloon 80 (e.g., FIGS. 12-13). In at least some embodiments, the syringe 10 may be as described herein. As such, some details thereof are not repeated. In some embodiments, the method of using the syringe 10 may comprise releasably engaging, releasably coupling, and/or releasably attaching the medical accessory 100 of FIGS. 2-5 to the syringe 10, as seen in FIG. 11. In some embodiments, the method of using the syringe 10 may comprise releasably engaging, releasably coupling, and/or releasably attaching the medical accessory 200 of FIGS. 8-10 to the syringe 10.

In some embodiments, the method of using the syringe 10 may comprise, with the medical accessory 100 (or the medical accessory 200) engaged with, coupled to, and/or attached to the barrel 20 of the syringe 10, retracting the plunger 30 disposed within the barrel 20 of the syringe 10 from a first position, wherein the stopper 32 is disposed adjacent a distal end of the barrel 20 of the syringe 10, to a second position spaced away from the distal end of the barrel 20 of the syringe 10 to fill the barrel 20 of the syringe 10 with an inflation fluid corresponding to an expanded size of the balloon 80. In some embodiments, the distal hub 50 of the syringe 10 may be fluidly coupled to and/or placed in fluid communication with a source of inflation fluid 90, using various means known in the art. Accordingly, said fluid communication is shown schematically in FIG. 11. In some embodiments, in the second position of the plunger 30, a distal edge of the stopper 32 (e.g., a distal end of a seal between the stopper 32 and the inner surface of the barrel 20) may be positioned at and/or in alignment with one of the plurality of markings 118/228/258 and/or one of the lines 119/229/259.

In some embodiments, after filling the barrel 20 of the syringe 10 with inflation fluid, and with the medical accessory 100 (or the medical accessory 200) still engaged with, coupled to, and/or attached to the barrel 20 of the syringe 10, the syringe 10 may be fluidly coupled to a balloon catheter 82. In some embodiments, the balloon catheter 82 may be configured to be advanced to a treatment site over a guidewire 84 with the balloon 80 in a deflated configuration. In some embodiments, the syringe 10 may be fluidly coupled to the balloon catheter 82 after advancing the balloon 80 to the treatment site. In some embodiments, the syringe 10 may be fluidly coupled to the balloon catheter 82 after advancing the balloon 80 to the treatment site.

In some embodiments, after filling the barrel 20 of the syringe 10 with inflation fluid, and with the medical accessory 100 (or the medical accessory 200) still engaged with, coupled to, and/or attached to the barrel 20 of the syringe 10, and/or with the syringe 10 fluidly coupled to the balloon catheter 82, the method of using the syringe 10 may comprise depressing the plunger 30 of the syringe 10 from the second position (e.g., FIG. 11) toward and/or to the first position to inflate the balloon 80 to an inflated configuration, as seen in FIG. 12.

In some embodiments, the method of using the syringe 10 may comprise subsequently retracting the plunger 30 of the syringe 10 within the barrel 20 of the syringe 10, with the medical accessory 100 (or the medical accessory 200) still engaged with, coupled to, and/or attached to the barrel 20 of the syringe 10, and/or with the syringe 10 fluidly coupled to the balloon catheter 82, toward and/or to a third position, wherein the stopper 32 is disposed proximal of the second position, to apply a vacuum to the balloon catheter 82 and/or the balloon 80, thereby deflating the balloon 80 to the deflated configuration. In some embodiments, the method of using the syringe 10 may comprise subsequently retracting the plunger 30 of the syringe 10 and/or the stopper 32 of the plunger 30 within the barrel 20 of the syringe 10, with the medical accessory 100 (or the medical accessory 200) still engaged with, coupled to, and/or attached to the barrel 20 of the syringe 10, and/or with the syringe 10 fluidly coupled to the balloon catheter 82, toward and/or to a proximal end of the barrel 20 of the syringe 10 to apply a vacuum to the balloon catheter 82 and/or the balloon 80, thereby deflating the balloon 80 to the deflated configuration. In some embodiments, the third position of the plunger 30 is wherein the stopper 32 is disposed at and/or adjacent the proximal end of the barrel 20 of the syringe 10.

As discussed herein, in some embodiments, the medical accessory 100 (or the medical accessory 200) may be configured to prevent the plunger 30 of the syringe 10 from exiting and/or being withdrawn from the barrel 20 of the syringe 10. In some embodiments, the lateral extension 134 of the medical accessory 100 (or the first lateral extension 234 and the second lateral extension 264 of the medical accessory 200) may be configured to prevent the plunger 30 of the syringe 10 from exiting and/or being withdrawn from the barrel 20 of the syringe 10.

The materials that can be used for the various components of the medical accessory and the various elements thereof disclosed herein may include those commonly associated with medical devices. For simplicity purposes, the following discussion refers to the device. However, this is not intended to limit the devices, components, and methods described herein, as the discussion may be applied to other elements, members, components, or devices disclosed herein, such as, but not limited to, the medical accessory, the syringe, etc. and/or elements or components thereof.

Some examples of suitable polymers may include polytetrafluoroethylene (PTFE), ethylene tetrafluoroethylene (ETFE), fluorinated ethylene propylene (FEP), polyoxymethylene (POM; for example, DELRIN®), polyether block ester, polyurethane, polypropylene (PP), polyvinylchloride (PVC), polyether-ester (for example, ARNITEL®), ether or ester based copolymers (for example, butylene/poly(alkylene ether) phthalate and/or other polyester elastomers such as HYTREL®), polyamide (for example, DURETHAN® or CRISTAMID®), elastomeric polyamides, block polyamide/ethers, polyether block amide (PEBA; for example, PEBAX®), ethylene vinyl acetate copolymers (EVA), silicones, polyethylene (PE), MARLEX® high-density polyethylene, MARLEX® low-density polyethylene, linear low density polyethylene (for example, REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID®), perfluoro(propyl vinyl ether) (PFA), ethylene vinyl alcohol, polyolefin, polystyrene, epoxy, polyvinylidene chloride (PVdC), poly(styrene-b-isobutylene-b-styrene) (for example, SIBS and/or SIBS 50A), polycarbonates, polyurethane silicone copolymers (for example, Elast-Eon® or ChronoSil®), biocompatible polymers, other suitable materials, or mixtures, combinations, copolymers thereof, polymer/metal composites, and the like. In some embodiments, the device and/or components thereof can be blended with a liquid crystal polymer (LCP).

Some examples of suitable metals and metal alloys include stainless steel, such as 304 and/or 316 stainless steel and/or variations thereof; mild steel; nickel-titanium alloy such as linear-elastic and/or super-elastic nitinol; other nickel alloys such as nickel-chromium-molybdenum alloys (e.g., UNS: N06625 such as INCONEL® 625, UNS: N06022 such as HASTELLOY® C-22®, UNS: N10276 such as HASTELLOY® C276®, other HASTELLOY® alloys, and the like), nickel-copper alloys (e.g., UNS: N04400 such as MONEL® 400, NICKELVAC® 400, NICORROS® 400, and the like), nickel-cobalt-chromium-molybdenum alloys (e.g., UNS: R30035 such as MP35-N® and the like), nickel-molybdenum alloys (e.g., UNS: N10665 such as HASTELLOY® ALLOY B2®), other nickel-chromium alloys, other nickel-molybdenum alloys, other nickel-cobalt alloys, other nickel-iron alloys, other nickel-copper alloys, other nickel-tungsten or tungsten alloys, and the like; cobalt-chromium alloys; cobalt-chromium-molybdenum alloys (e.g., UNS: R30003 such as ELGILOY®, PHYNOX®, and the like); platinum enriched stainless steel; titanium; platinum; palladium; gold; combinations thereof; or any other suitable material.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The scope of the disclosure is, of course, defined in the language in which the appended claims are expressed.

What is claimed is:

1. A medical accessory for use with a syringe, comprising:
a body portion configured to releasably couple to a barrel of the syringe; and
a proximal flange portion configured to engage with a finger flange of the syringe.

2. The medical accessory of claim 1, wherein the body portion comprises a window configured to permit viewing of the barrel of the syringe through the body portion.

3. The medical accessory of claim 2, wherein the body portion comprises a plurality of markings corresponding to different fluid volumes within the barrel of the syringe disposed along the window.

4. The medical accessory of claim 3, wherein the different fluid volumes correspond to different balloon inflation sizes.

5. The medical accessory of claim 2, wherein the body portion comprises a proximal portion extending distally from the proximal flange portion and a distal portion disposed distal of the window.

6. The medical accessory of claim 5, wherein the body portion extends circumferentially around a central longitudinal axis; and
wherein the proximal portion extends a first angular distance around the central longitudinal axis and the distal portion extends a second angular distance around the central longitudinal axis greater than the first angular distance.

7. The medical accessory of claim 6, wherein the first angular distance is greater than 180 degrees and less than 270 degrees.

8. The medical accessory of claim 1, wherein the body portion is configured to snap onto the barrel of the syringe.

9. The medical accessory of claim 1, wherein the body portion is configured to engage the syringe in a lateral direction.

10. The medical accessory of claim 1, wherein the body portion comprises a C-shaped shell configured to engage a cylindrical outer surface of the barrel of the syringe.

11. The medical accessory of claim 1, wherein the proximal flange portion is configured to engage the finger flange of syringe in a lateral direction.

12. The medical accessory of claim 11, wherein the proximal flange portion is configured to slidably receive the finger flange of the syringe within an interior cavity of the proximal flange portion.

13. The medical accessory of claim 1, wherein the proximal flange portion comprises a lateral extension extending from the proximal flange portion toward a central longitudinal axis of the body portion, the lateral extension being configured to prevent a plunger of the syringe from being removed from the barrel of the syringe.

14. A medical accessory for use with a syringe, comprising:
a first portion comprising a first body portion configured to releasably couple to a barrel of the syringe, and a first proximal flange portion configured to engage with a finger flange of the syringe; and
a second portion comprising a second body portion configured to releasably couple to the barrel of the syringe, and a second proximal flange portion configured to engage with the finger flange of the syringe;
wherein the first portion is hingedly attached to the second portion.

15. The medical accessory of claim 14, wherein the first proximal flange portion comprises a first lateral extension extending toward a central longitudinal axis of the medical accessory and the second proximal flange portion comprises a second lateral extension extending toward the central longitudinal axis of the medical accessory.

16. The medical accessory of claim 15, wherein the first lateral extension and the second lateral extension are configured to prevent a plunger of the syringe from being removed from the barrel of the syringe.

17. The medical accessory of claim 14, wherein the first body portion comprises a first window configured to permit viewing of the barrel of the syringe through the first body portion, and the second body portion comprises a second window configured to permit viewing of the barrel of the syringe through the second body portion.

18. The medical accessory of claim 17, wherein the first window has a first length extending longitudinally along more than 50% of a length of the first body portion, and the second window has a second length extending longitudinally along more than 50% of a length of the second body portion.

19. The medical accessory of claim 14, wherein the first body portion is configured to engage with the second body portion to collectively surround the barrel of the syringe.

20. A method of using a syringe in a procedure involving inflation and deflation of a balloon, comprising:
releasably coupling a medical accessory to the syringe, the medical accessory comprising a body portion configured to releasably couple to a barrel of the syringe, and a proximal flange portion configured to engage with a finger flange of the syringe;
with the medical accessory coupled to the barrel of the syringe, retracting a plunger disposed within the barrel of the syringe from a first position adjacent a distal end of the barrel of the syringe to a second position to fill the barrel of the syringe with an inflation fluid corresponding to an expanded size of the balloon;
after filling the syringe with inflation fluid, and with the medical accessory still coupled to the barrel of the syringe, depressing the plunger of the syringe from the second position to the first position to inflate the balloon; and
subsequently retracting the plunger of the syringe, with the medical accessory still coupled to the barrel of the syringe, to a proximal end of the barrel of the syringe to apply a vacuum to the balloon, thereby deflating the balloon;
wherein the medical accessory is configured to prevent the plunger of the syringe from exiting the barrel of the syringe.
